SEQUENCE LISTING (9 sequences)

SEQ ID NO: 1 - protein, 7 aa, Homo sapiens
APLILSR

SEQ ID NO: 2 - protein, 7 aa, Homo sapiens
PLILSRI

SEQ ID NO: 3 - protein, 6 aa, Homo sapiens
PLILSR

SEQ ID NO: 4 - protein, 5 aa, Homo sapiens
LILSR

SEQ ID NO: 5 - protein, 4 aa, Homo sapiens
ILSR

SEQ ID NO: 6 - protein, 7 aa, Homo sapiens
VPLIGSR

SEQ ID NO: 7 - protein, 6 aa, Homo sapiens
LILSRI

SEQ ID NO: 8 - protein, 5 aa, Homo sapiens
ILSRI

SEQ ID NO: 9 - protein, 4 aa, Homo sapiens
LSRI

(19) United States
(12) Patent Application Publication
(10) Pub. No.: US 2004/0214246 A1
(43) Pub. Date: Oct. 28, 2004

(54) Antibodies against particular forms of propsa and use thereof in immunoassays

(76) Inventors: Wolfgang Hoesel, Tutzing (DE); Jochen Peter, Research Triangle Park, NC (US); Ole Vorm, Odense (DK); Thomas N. Krogh, Odense S (DK)

Correspondence Address: Roche Diagnostics Corporation, 9115 Hague Road, PO Box 50457, Indianapolis, IN, 46250-0457, US

(21) Appl. No.: 10/296,272

(22) PCT Filed: May 23, 2001
(86) PCT No.: PCT/EP01/05943

(30) Foreign Application Priority Data
May 24, 2000 (DE) 10025387.3
Jul. 5, 2000 (DE) 10032040.6

(51) Int. Cl. G01N 33/574; C07K 16/30

(52) U.S. Cl. 435/7.23; 530/388.8

(57) ABSTRACT

The invention concerns antibodies to special forms of enzymatically non-active precursors of prostate specific antigen (PSA), their production, their use in immunoassays for determination of [−5, −6, −7] proPSA and the use of measured values from these assays for improved diagnostics in the field of prostatic carcinoma.

### FIELD OF THE INVENTION

The invention concerns antibodies to special forms of the enzymatically inactive precursors of the prostate-specific antigen (PSA), their manufacture, their application in immunoassays for determination of [−5, −6, −7] proPSA, and the use of measured values from these assays for improved diagnostics in the field of prostate cancer.

### BACKGROUND OF THE INVENTION

Carcinoma of the prostate (PCa) is the most frequently diagnosed form of cancer in men in the United States (Parker, S. L. et al., CA Cancer J. Clin. 46:5-27, 1996). The prostate-specific antigen, also referred to as PSA, was used to a large extent as a reliable prognosis marker for the treatment of patients with prostate cancer (Catalona, W. J. et al., N. Engl. J. Med. 324:1156-1161, 1991; Osterling, J. E., J. Urol. 145:907-923, 1991). As a tumor marker it has the considerable advantage that it is not detectable in the blood of healthy men, or, if it is, only in very low concentrations. Highly elevated PSA values are generally measured in advanced stages of the disease.

A clinical problem of enormous significance is the early, certain detection of PCa and the differentiation of PCa and malignant prostatic hyperplasia (BPH). One of the main problems with current PSA tests is the fact that this differentiation between BPH and PCa is not ensured (McCormack, R. T. et al., Urology 45:729-744, 1995). For example, with PSA concentrations in the range of 2-15 ng/ml serum, the presence of a malignancy cannot be definitively determined because such PSA values can also be caused by BPH. Expensive and painful tests (e.g., rectal biopsies) must be performed in addition to PSA determination in order to clarify the presence of PCa.

In order to improve the diagnostic accuracy of PSA detection in serum, various alternative procedures have been proposed, such as PSA density, PSA rate, ratio of free to total PSA, or between complexed and total PSA (Benson, M. C. et al., J. Urol. 147:815-816, 1992; Carter, H. B. et al., J. Am. Med. Assoc. 267:2215-2220, 1992; Oesterling, J. E. et al, J. Am. Med. Assoc. 270:860-864, 1993).

PSA belongs to a group of proteins/enzymes known as “kallikreins”. The human kallikrein family is composed of three members, hK1, hK2 and hK3, or PSA (Clements, J. A., Endocr. Rev. 10:393-419, 1989; Carbini, L. A. et al., J. Hypertens. 11:893-898, 1993).

hK1 is produced mainly in the kidneys, pancreatic ducts and the submandibulary salivary glands (Fukushima, D. et al., Biochemistry 24:8037-8043, 1985).

hK2, like PSA, is produced primarily in the prostatic epithelium (Morris, B. J., Clin. Exp. Pharm. Phys. 16:345-351, 1989) and has 78% homology with PSA (Schedlich, L. J. et al., DNA 6:429-437, 1987; Lilja, H., World J. Urol. 11:188-191, 1993). The properties of hK2 as a potential prostate cancer marker were discussed in an overview article (Young, C. Y. F. et al., The Prostata Supplement 7: 17-24, 1996).

The prostate-specific antigen (PSA), or hK3, is a glycoprotein with a molecular weight of approximately 29,000 daltons. It is formed in the prostatic epithelial cells and is a component of seminal fluid. PSA has the enzymatic activity of a neutral serine protease. Its main function is to cleave seminogelins I and II and fibronectin, which, as essential components of ejaculate, block the mobility of sperm. By hydrolyzing these proteins, PSA brings about the liquification of the seminal coagulum, which allows the mobility of sperm.

PSA is a chymotrypsin-like protease, while hK2 is a trypsin-like protease.

It is known that PSA occurs in different molecular forms in serum. The greatest portion by far of PSA occurs in the form of inactive complexes, whereby, above all, α1-anti-chymotrypsin complexes PSA, thereby inhibiting its enzymatic activity as well.

Further complexes can be formed with other serine protease inhibitors (serpins), such as α1-antitrypsin and protein C inhibitors which are present in the serum only to a very small extent as compared to PSA-ACT. Moreover, PSA also forms a complex with another type of protease inhibitor, α2-macroglobulin (α2-M). Varying statements have been made in the literature about the content of this protease inhibitor in serum, mainly since PSA is not immunologically accessible in this complex. The expression “total PSA” used herein refers to the sum of free PSA and PSA complexed with serpins, since PSA complex with α2-M cannot be detected using any immunological determination methods used to date (Tewari and Bluestein, J. Clin. Ligand Assay 18:186-196, 1995). In addition to “total PSA”, the expression “PSA total” is also used with the same meaning.

Similarly, the same definition applies for total kallikrein for the other two kallikreins, hK1 and hK2. It is often referred to as kallikrein (total) or PSA (total).

In addition to complexed PSA, enzymatically inactive, free PSA that cannot enter into complexes is also present in the serum. Petterson et al., Clin. Chem. 41:1480-1488, 1995, describe that approximately 15% to 20% of the total PSA in free (non-complexed) form is present in serum. Petterson et al. and other working groups have therefore discussed the fact that this free, enzymatically non-active PSA may represent a pro-enzyme form or it may have been rendered inactive by internal cleavage of the lys-lys bonds between amino acids 145 and 146 of PSA.

The PSA molecule is synthesized as a pre-proform consisting of 261 amino acids. The 17 amino acid-long signal peptide is cleaved in the endoplasmatic reticulum. Accordingly, the enzymatically non-active proPSA (zymogen) is still 244 amino acids long.

The enzymatically active, mature PSA results when the propeptide (7 amino acids) is cleaved (Melegos and Diamandis, Clin. Biochem. 29:193-200, 1996).

The proPSA molecule with the 7 N-terminal amino acids of PSA propeptide is also referred to as [−7] proPSA. ProPSA molecules with an N-terminally shortened sequence are referred to as [−6], [−5], [−4] etc. proPSA, depending on the length of the propeptide portion.

The exact structure of the forms of PSA occurring in human serum has not yet been determined or analyzed in detail. Above all, for instance, the appearance of the exact structure of PSA occurring freely in serum, i.e., not in complexes, has not yet been clarified (Paus et al., J. of Urology 159:1599-1605, 1998). The reason is that the concentration of free PSA in serum is so low that it is very difficult to isolate enough material and make it available for laboratory testing. Noldus et al., J. of Urology 158:1606-1609, 1997, isolated free PSA in impure form with a yield of approximately 25% from 230 ml of a mixture of high-titer PSA sera (PSA total>2000 ng/ml) and analyzed it using N-terminal sequencing. They found indications of the presence of a normal N-terminal and cleavage of the PSA chain between amino acids 145 and 146. This may explain that this PSA is not present in complexed form. They found no indications of proPSA forms, i.e., PSA molecules with additional amino acids on the N-terminal end.

In WO 98/49323, antibodies to PSA were used to identify and molecularly characterize the molecular forms of free PSA occurring in biological samples. It was demonstrated that a significant portion of free PSA occurs in serum as [−4] proPSA, i.e., as an N-terminal PSA molecule with an extension of 4 amino acids per proPSA sequence.

The related art does not include any specific diagnostic methods for free PSA molecules that still contain portions of the propeptide. Just as poorly understood is whether proPSA forms other than the [−4] proPSA described in WO 98/49323 occur in sample fluids of human origin. Additionally, the related art includes no data on the diagnostic value of free PSA having proPSA sequence portions of different lengths.

### SUMMARY OF THE INVENTION

The object of the invention, therefore, is to provide diagnostic tools for performing specific detection of certain proPSA forms and to determine if these novel tests can contribute to improved diagnostics in the field of prostatic carcinoma, in particular in the differentiation of BPH and PCa.

In an initial step, the latest analytical methods were therefore used to determine if indications of other proPSA forms could be found that do not correspond to the [−4] proPSA from WO 98/49323. Surprisingly, indications of at least three additional proPSA forms, [−2, −5, −7] proPSA, were found. Based on these preliminary tests, the question about the diagnostic relevance of these proPSA forms was investigated.

In order to perform the work necessary to attain the object of the invention, antibodies were produced and specifically selected that react with [−5, −6, −7] proPSA but do not significantly detect shorter proPSA forms, [−4], [−3] etc.

Synthetic peptides that correspond to the N-terminal sequences of proPSA forms having different lengths were produced and used for purposes of immunization, screening and characterization.

Antibodies were developed and isolated that have specificities relevant to the invention. Such antibodies react with [−5, −6, −7] proPSA but they exhibit no significant binding properties to peptides of [−4] proPSA or shorter PSA peptides.

A preferred exemplary embodiment, therefore, comprises antibodies to proPSA, characterized by the fact that they bind to [−5, −6, −7] proPSA but basically do not react with [−4] proPSA or further shortened proPSA forms.

These antibodies were purified, modified, and derivatized in the usual fashion and used in immunoassays to detect [−5, −6, −7] proPSA.

Surprisingly, it was observed that native samples contain a measurable portion of [−5, −6, −7] proPSA. Moreover, it was also surprisingly observed that these special proPSA forms are relatively stable and even occur in the samples investigated in such a significant concentration that they can be measured using immunoassays.

The [−5, −6, −7] proPSA values determined using the methods according to the invention were compared with PSA assays known from the prior art in terms of clinical and diagnostic relevance.

As mentioned initially, one of the main problems with the use of PSA measurements in clinical diagnostics is the range of low, slightly elevated PSA values. A differentiation between BHP and PCa cannot be performed with total PSA values. The test, according to the invention, for special forms of proPSA provides important and diagnostically relevant additional information in this clinically particularly critical decision range, i.e., with total PSA concentrations below 20 ng/ml, e.g., determined with the ELECSYS® test for total PSA from Roche Diagnostics, therefore making an important contribution to the improved differentiation between PCa and BPH.

A further preferred embodiment of the invention, therefore, is the use of [−5, −6, −7] proPSA values for diagnostics-related clinical problems in the field of prostatic carcinoma.

With the antibodies according to the invention and the immunological methods established using them, it is possible for the first time to calculate quotients of the total amount of free PSA and [−5, −6, −7] proPSA. Since it was surprisingly determined that this quotient substantially improves the clinical/diagnostic findings, the use of a quotient of free PSA to [−5, −6, −7] proPSA therefore represents a particularly preferred embodiment of the invention.

### DESCRIPTION OF THE DRAWINGS

FIG. 1: ProPSA peptides from the MALDI-TOF analysis

Free PSA was isolated from PCa plasma using immunosorption, separated on an SDS gel, and the free PSA bands were digested with Endo Lys C. 0.5 μL of this sample were mixed on the MALDI sample tray with 0.5 mL matrix (10 mg/ml sinapic acid in 0.1% TFA:acetone nitrile, 7:3, v:v), and the mass spectrum was determined. The descriptions of the peptides above the mass numbers, e.g., 114-145, indicates the range of the amino acid sequence of PSA. The peptide corresponding to the [M+H]⁺ ion peak 1939.10 is the incompletely cleaved peptide with the amino acid range 222-237 of PSA. The pro sequence of PSA extends from the amino acid position −7 to −1.

FIG. 2: Competition test with free proPSA peptides

ProPSA from a PCa plasma was bound via a biotinylated antibody to free PSA to a solid phase coated with streptavidin. The binding or displacement of MAB 1.023.6 by proPSA peptides of different lengths was determined and is shown in this figure.

FIG. 3: ROC analysis of clinical relevance of various PSA parameters

The figure shows the AUC (area under the curve) values calculated via ROC analysis. The greater the AUC value, the better a parameter, or ratio of parameters, is in terms of clinical accuracy and clinical relevance with regard for sensitivity and specificity.

### DETAILED DESCRIPTION OF THE INVENTION

The precursor of the PSA molecule is pre-proPSA, which is composed of 261 amino acids. The signal peptide is 17 amino acids long and is cleaved in the endoplasmatic reticulum as soon as pre-proPSA enters the secretory ducts. The enzymatically inactive proPSA (this inactive precursor of PSA is also called a zymogen) that is produced is converted to the enzymatically active, mature PSA via release (cleavage) of the seven N-terminal amino acids of the propeptide. This mature, enzymatically active form has 237 amino acids and a molecular weight of approximately 29 kD.

It is known that PSA, as well as the two other kallikreins hK1 and hK2, are present in circulation largely in the form of inactive complexes, i.e., bound to various protease inhibitors. The greatest portion by far is complexed with α1-anti-chymotrypsin.

The propeptide of [−7] proPSA is 7 amino acids long, as described above. It has the amino acid sequence alanine-proline-leucine-isoleucine-leucine-serine-arginine (APLILSR). The propeptide sequence for hK2 is valine-proline-leucin-isoleucin-glutamine-serin-arginine (VPLIQSR). The first N-terminal amino acid of PSA is an isoleucine. It was determined, surprisingly, that antibodies having the reaction spectrum according to the invention, i.e., good binding to the proPSA forms [−5], [−6] and [−7] can be obtained when a synthetic peptide that corresponds to the [−6] proPSA is used for immunization. The peptide actually used also contains the first amino acid from the mature PSA. Its sequence, therefore, is PLILSRI.

It is known that short peptides are only slightly or not immunogenic per se. For purposes of immunization, it is therefore usually necessary to couple peptides to suitable carrier structures, e.g., bovine serum albumin or keyhole limpet hemocyanin. If necessary, the immune response is also strengthened by adding “adjuvant reagents”. A preferred embodiment of the invention is therefore immunogens that contain the [−5, −6, −7] propeptide of PSA. Immunogens that can be obtained by using the peptide of SEQ ID No: 2 are particularly preferred.

The antibodies according to the invention can also be obtained using suitable combinations of other immunogens, such as a [−7] proPSA peptide or mimetopes, e.g., of the [−6] proPSA with the screening criteria described below.

An object of the invention, therefore, is antibodies with specificity for [−5, −6, −7] proPSA that are essentially non-reactive with [−4] proPSA and shorter PSA forms. The different proPSA forms [−7], [−6] and [−5] proPSA are bound effectively by such antibodies, while the [−4] proPSA is essentially non-reactive.

The PLILSRI peptide was produced synthetically using standard methods (Peptide, Hans-Dieter Jakubke, Spektrum Verlag, Heidelberg, 1996). Experience has shown that short peptides as such are not very immunogenic. For purposes of immunization, the synthetic PLILSRI peptide is therefore bound to a suitable carrier protein, such as keyhole limpet hemocyanin, bovine serum albumin or edestin.

Immunization is performed in the laboratory animals typically used for this purpose, i.e., mice, rats, rabbits or sheep. Typical adjuvant substances, e.g., Freund's adjuvant, are used to stimulate the immune response.

To produce monoclonal antibodies, spleen cells, preferably from mice, the animals having been immunized accordingly, are immortalized using methods known to one skilled in the art. Particularly preferred are methods based on the hybridoma technique (Köhler and Milstein, Nature 256:495-497, 1975) or transformation with the Eppstein-Barr virus (EBV transformation) (Monoclonal Antibody Production Techiques and Application, Lawrence B. Schook, Ed., Marcel Dekker Verlag, 1987).

The quality of the immune response fluctuates greatly from animal to animal and from species to species. For this reason it is necessary to use suitable screening methods that ensure that the antibodies obtained also have the necessary properties.

A further object of the invention, therefore, is a production method for antibodies that includes a suitable screening system. This screening system ensures that the antibodies obtained via the immunization described above have suitable binding properties to [−5, −6, −7] proPSA and are essentially non-reactive with [−4] proPSA and shorter proPSA forms.

A particularly suitable screening system contains the 7 amino acid-long PLILSRI peptide that is coated on streptavidin solid phases in biotinylated form. As an alternative, PSA propeptide can also be bound to proteins and adsorbed on solid phases via said proteins. In all of these screening systems, such variants are particulary preferably used that ensure that the carrier protein as well as the coupling chemistry differ from the ingredients used to produce the immunogen, i.e., it is particularly preferable to use alternative carrier proteins and other types of linker structures.

A further particularly suitable screening system is a competitive immunoassay that uses biotinylated PSA (−6 to +1) propeptide (PLILSRI-Bi) as the antigen. In this test system, the biotinylated peptide is coated via streptavidin on the solid phase. An investigation is then carried out to determine the extent to which other/shortened peptides from the propeptide region of PSA in this test can displace the antibodies according to the invention. Antibodies are selected that react with [−5, −6, −7] proPSA but basically do not detect [−4] proPSA.

An alternative screening system that can also be used in converted form (see below) to detect proPSA is a sandwich test. In this test, PSA, e.g., from sperm, and proPSA and PSA from PCa plasma are tested as antigens.

The binding of the proPSA to the streptavidin-coated solid phase takes place via the F(ab) fragments, by an antibody to free PSA. The new antibodies to proPSA to be tested are used for detection. In an initial screening, antibodies are selected that are reactive with the proPSA from the PCa serum and that do not react with PSA from sperm.

The system described above can further be used to select out the antibodies according to the invention via suitable displacement reactions. For this purpose, proPSA from PCa serum is bound to the solid phase as described above. Further specificity testing is then carried out as described above for tests with biotinylated proPSA peptide as the antigen.

A further object of the invention, therefore, are antibodies that exhibit good competition, in the competition test described above, with the [−7] PSA propeptide, with the [−6] PSA propeptide and with the [−5] PSA propeptide, but basically do not compete with the [−4] PSA propeptide. Competition in this contexts means that the [−5, −6, −7] proPSA peptides compete effectively with the antibodies under investigation to bind to the PLILSRI peptide on the solid phase. Basically non-reactive antibodies to [−4] proPSA and shorter proPSA sequences are antibodies that, in the competition test, exhibit less than 10% of the displacement effect, particularly preferred less than 5%, and most preferably preferred, less than 3% of the displacement effect as compared with a [−5] proPSA peptide.

The term “antibodies” in the context of the invention refers to the intact immunoglobulins as well as all antibody fragments. These include, for example, Fab, Fab′ or F(ab)′₂ fragments. The term “antibody” without the descriptor “monoclonal” or “polyclonal” always includes both types of antibodies, as well as any chimeric constructs and all fragments listed above.

Antibodies that basically exhibit the same binding properties are understood to be antibodies that compete immunologically with the clone stored on May 16, 2000 as ACC 2456 at the Deutschen. Sammlung für Mikroorganismus und Zellkulturen (DSMZ) in Braunschweig, to bind to [−5, −6, −7] proPSA.

A particularly preferred embodiment of the invention comprises antibodies that basically have the same binding properties as the clone stored as ACC 2456 at the DSMZ.

The antibodies according to the invention can be used to specifically detect [−5, −6, −7] proPSA. Immunological methods based on the competition principle or the sandwich principle are particularly preferred. The multifarious embodiments of immunoassays are known to one skilled in the art, so a detailed description will not be provided here.

A further object of the invention, therefore, are immunological test procedures that are suited for the quantification/detection of [−5, −6, −7] proPSA in a sample.

All biological fluids common to one skilled in the art can be used as samples. Bodily fluids such as whole blood, blood serum, blood plasma and urine are preferred as samples.

The [−5, −6, −7] proPSA is detected particularly preferrably in a sandwich test. It has proved particularly advantageous to use a specific antibody to [−5, −6, −7] proPSA as a specific capture antibody. “Specific” is understood to mean, in this context, that the capture antibody fulfills the specificity requirements described in the screening section.

A further object of the invention, therefore, is the use of antibodies to [−5, −6, −7] proPSA in immunoassays to detect these molecules.

Furthermore, it has proven particularly advantageous to use an antibody in the detection of [−5, −6, −7] proPSA that reacts specifically with free PSA. An antibody having specificity for free PSA is understood to mean that such an antibody does not detect PSA molecules that are complexed with α1-anti-chymotrypsin (ACT).

The level of [−5, −6, −7] proPSA proved to be easy to measure. Using the test system described above, critical human serum, i.e., human sera with approximately 4 to approximately 10 ng/ml total PSA were therefore investigated. An analysis of the measured values showed that [−5, −6,

−7] proPSA values provide valuable additional information in the clinically relevant and critical decision range. [−5, −6, −7] proPSA values can be used to diagnose or rule out the presence of a prostatic carcinoma.

As initially mentioned, a markedly elevated PSA value of >20 ng/ml has very high clinical/diagnostic relevance. Such a value is a comparably certain diagnostic indication of the presence of a PCa. The clinical relevance drops considerably when values fall below 20 ng/ml. A value below 10 ng/ml can be due to BPH with as much certainty as it can be due to a PCa. In this range in particular, it would be particularly important to differentiate a malignant prostate hyperplasia from a PCa in the early stage.

An inclusion criterium for the selected sera were a PSA (total) value of 4-10 ng/ml that was determined with the appropriate ELECSYS test from Roche Diagnostics. Free PSA was determined in addition to PSA (total) using the appropriate ELECSYS test from Roche Diagnostics as well.

The clinical relevance of the tested PSA markers was investigated individually and in relation to each other in the clinically particularly relevant range (4-10 ng/ml) using ROC analyses.

An ROC analysis (ROC=receiver operator curve) refers to the calculation and compilation of a sensitivity-specificity diagram. A test with good diagnostic separation efficiency has the greatest possible AUC value (AUC=area under the curve). The higher this value, the better the performance features of the evaluated test in terms of its specificity and sensitivity.

The ratio of [−5, −6, −7] proPSA to free PSA was first imaged and evaluated as a result of the newly developed immunoassay. Surprisingly, this ratio proved to have particular clinical relevance in the ROC analyses performed, i.e., using the ratio of [−5, −6, −7] proPSA to free PSA, a better differentation of PHB and PCa can be performed than using the parameters known in the related art. This advantage becomes apparent in FIG. 3.

A further preferred embodiment of the invention, therefore, is the more in-depth analysis of critical PSA sera, i.e., sera having a total PSA of less than approximately 20 ng/ml, particularly preferably with a PSA of 2-15 ng/ml and, particularly, of 4-10 ng/ml by evaluating the quotient of [−5, −6, −7] proPSA to free PSA or the inverse quotient.

The antibodies according to the invention can also be used to determine the three different proPSA forms, [−5] proPSA, [−6] proPSA and [−7] proPSA, individually. For this purpose, an antibody according to the invention is used to enrich the [−5], [−6] and [−7] proPSA molecules from the sample and make them accessible for further analysis. Particularly preferably, the enrichment takes place using immunoaffinity chromatography or selective immunosorption, and the individual detection of the three proPSA forms described above is performed using methods based on mass spectrometry (MS).

While the immunological method according to the invention detects the proPSA forms [−5, −6, −7] in total, the three proPSA forms can be quantified individually using MS.

A further object of the invention, therefore, is the separate quantification of [−5], [−6] and [−7] proPSA and the use of these individual values to diagnose or rule out a prostatic carcinoma.

The antibodies according to the invention also react with [−6] prohK2, with the amino acid sequence proline-leucine-isoleucine-glutamine-serine-arginine (PLIQSR). Analogous to the detection methods and analyses described above, prohK2 can also be determined and used for diagnostic purposes using the antibodies according to the invention.

The amount of the three proPSA forms [−5], [−6] and [−7] can also be compared with the basic diseases and courses in order to distinguish an aggressive course of the disease form a slower course of the disease.

The following examples further explain the invention.

### EXAMPLE 1

Individual Detection of Different ProPSA Forms Using Mass Spectroscopy

a) Isolation of Free PSA from a PCa Serum Using Immunosorption

4 ml of a solution of the antibody MAB<PSA>-M-30-IgG-biotin (c=25 μg/ml) in PBS, pH 7,+1% BSA+0.1% TWEEN 20 were added to 28.7 mg magnetic streptavidin beads in a 10 ml vial and incubated for one hour. The beads were then precipitated using a magnet, the supernatant was removed via pipette, and the beads were washed three times with 3 ml wash solution (PBS, pH 7, +20 mM octylglucoside) each. After adding 8 ml plasma (content of free PSA=180 ng/ml), the solution was incubated for another 60 minutes at room temperature. The plasma was then removed via pipette, and the beads were washed 4× with 1 ml wash solution each time. 500 μl propionic acid, 1 M, were then added to the beads, and the suspension was shaken again for one hour. The supernatant was removed after precipitation of the beads, lyophilized, and then added to 10 μl distilled water. PSA from sperm was obtained from Scripps Laboratories, San Diego.

b) Identification of Free ProPSA Forms Using Matrix-Assisted Laser Desorption-Time of Flight Mass Spectrometry (MALDI-TOF MS)

An initial indication that the immunosorptively purified, free PSA from plasma differs in terms of size from that obtained from sperm was demonstrated using MALDI-TOF MS. It was determined that the free PSA from the PCa plasma with 29100 Da had a molecular weight that was higher than that of the free PSA from sperm by approximately 600 Da (28500 Da).

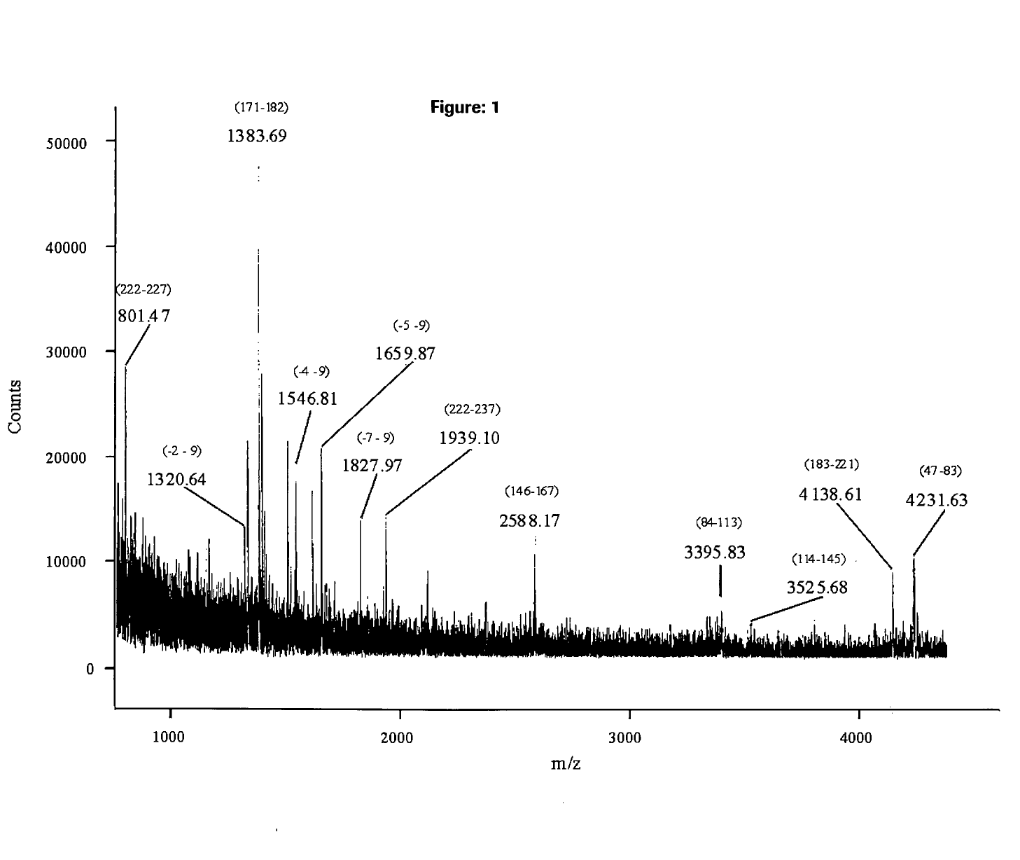

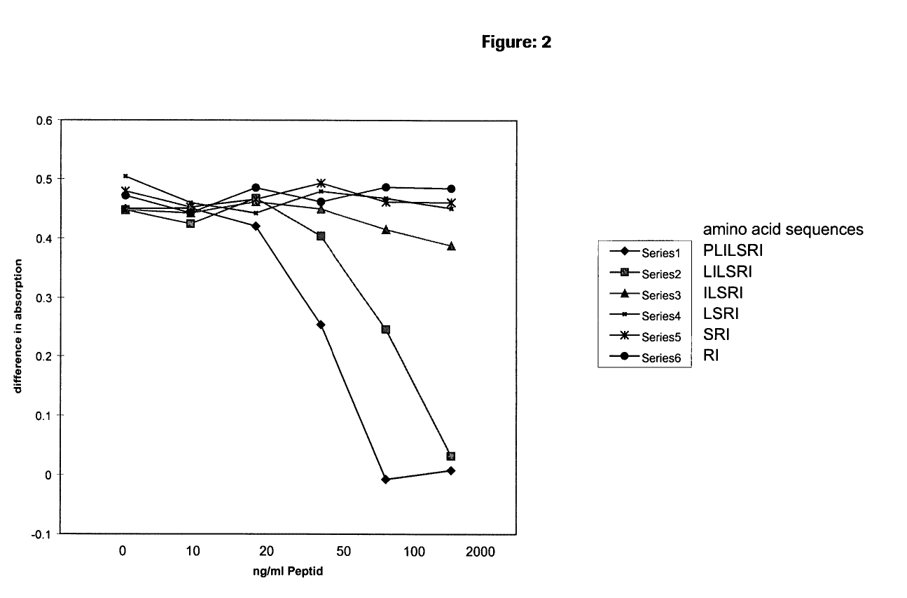

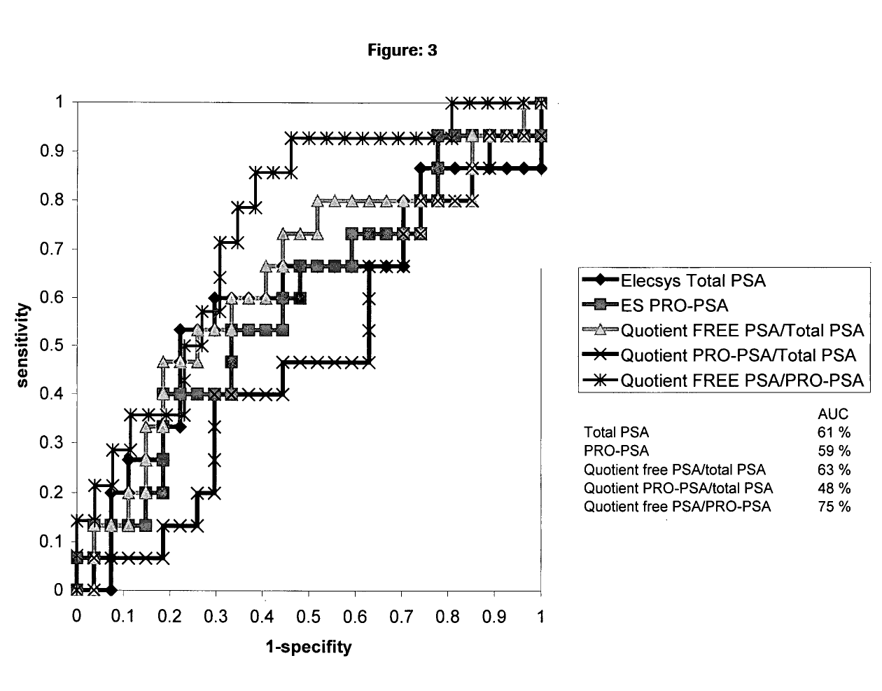

After separation of the free PSA from PCa plasma and sperm using SDS-PAGE, reductive alkylation of the bands and digestion with endoprotease Lys-C, the peptide pattern shown in FIG. 1 was obtained. For further clarification, the peptides detected in the respective PSA forms are summarized in Table 1.

TABLE 1
proPSA peptides from MALDI-TOF analysis

| [M + H]⁺ | [M + H]⁺ ions of | | |
|---|---|---|---|
| ions of peptides | peptides from | Theoretical values | |
| from free PSA | reference | of [M + H]⁺ ion | Amino acid |
| from plasma | PSA (sperm) | masses | sequence |
| Values in daltons | Values in daltons | Values in daltons | (from-to) |
| 801.47 | 801.50 | 801.96 | 222-227 |
| | 1077.54 | 1077.22 | 1-9 |
| 1321.64 | | 1320.49 | (−2)-9 |
| 1383.68 | 1384.35 | 1383.65 | 171-182 |
| 1546.80 | | 1546.81 | (−4)-9 |
| 1659.87 | | 1659.97 | (−5)-9 |
| 1827.97 | | 1828.16 | (−7)-9 |
| 1940.10 | | 1940.29 | 222-237 |
| 2588.23 | 2589.50 | 2587.98 | 146-167 |
| 3395.83 | 3397.83 | 3397.83 | 84-113 |
| 3525.68 | 3527.00 | 3525.95 | 114-145 |
| 4138.61 | 4141.71 | 4137.59 | 183-221 |
| 4231.63 | 4236.75 | 4234.86 | 47-83 |

As illustrated in FIG. 1 and Table 1, free PSA from PCa plasma contains four peptides that are not present in PSA from sperm. They are peptides that are extended by 2, 4, 5 or 7 amino acids on the N-terminal of PSA and therefore represent proPSA forms.

### EXAMPLE 2

Production of Monoclonal Antibodies (MAB) to ProPSA

a) Immunization of Mice

12-week old, female Balb/C mice were initially stimulated in an intraperitoneal manner with 100 μg of the peptide P-L-1-L-S—R-1-C (referred to hereinafter as proPSA peptide) that was coupled to cysteine via a spacer on KLH (keyhole limpet hemocyanin), together with an adjuvant (complete Freund's adjuvant). Three further intraperitoneal immunizations were performed after 6 weeks and then at monthly intervals. Each mouse was administered 100 μg of the immunogen described above, together with incomplete Freund's adjuvant (IFA). The final immunizations were then performed intravenously with 100 μg each of the immunogen in PBS buffer on the third, second and final day before fusion.

b) Fusion and Cloning

The fusion of spleen cells from the mice immunized as described above with myeloma cells was carried out as described by Galfré, Methods in Enzymology 73:3, 1981. In this procedure, approximately 1×10⁸ spleen cells from the immunized mice were mixed with 2×10⁷ myeloma cells (P3X63-Ag8-653, ATCC CRL1580) and centrifuged off (10 minutes at 300 g and 4° C.). The cells were then washed once with RPMI-1640 medium without fetal calf serum (FCS) and centrifuged once more at 400 g in a 50 ml cone vial. 1 ml PEG (polyethylene glycol, molecular weight 4000, Merck, Darmstadt) was added and mixed via pipetting. After 1 minutes in the water bath at 37° C., 5 ml RPMI 1640 without FCS was added in drops, mixed, filled to 50 ml with medium (RPMI 1640+10% FCS), then centrifuged. The sedimented cells were added to RPMI 1640 medium with 10% FCS and sowed in hypoxanthine-azaserine selection medium (100 mmol/l hypoxanthine, 1 μg/ml azaserine in RPMI 1640+10% FCS). Interleukin 6 (100 U/ml) was added to the medium as a growth factor. After 10 days, the primary cultures were tested for specific antibody synthesis.

c) Screening Test for Anti-ProPSA Peptide Antibodies

For screening mouse sera, primary cultures and the cloned MABs, microtiter plates coated with recombinant streptavidin (MicroCoat, Penzberg, Cat No. 12-K 96 N, lot MC 289) were coated with 1 μg/ml biotinylated proPSA peptide in PBS plus 0.5% Crotein C (100 μl per well, 10 minutes incubation at room temperature while shaking), and then washed 3× with 0.9% NaCl/0.05% TWEEN 20. In this conjugate, the proPSA peptide (PLILSRIC-Bi) was bound with biotin with another spacer than had been used with the immunogen described above to immunize mice in order to prevent spacer-specific antibodies. 100 μl of the antibody solution to be investigated was then added to a coated well and incubated for 1 hour at room temperature while shaking. After washing 3× with 0.9% sodium chloride/0.05% TWEEN 20, 100 μl of a peroxidase (POD)-labeled Fab fragment of a polyclonal antibody from sheep to mouse-Fcγ (Roche Diagnostics GmbH, I.D. No. 1431323, corresponding to 25 mU/ml) was added in each case to detect bound antibody in the sample, incubated for 1 hour at room temperature, and then washed 3× with 0.9% sodium chloride/0.05% TWEEN 20.

Finally, 100 μl ABTS® (Roche Diagnostics GmbH, Cat No. 1204521 and 1204530) was added to each well. After 30 minutes at room temperature, absorbance was measured at 405/492 nm in a MR700 microplate reader from Dynatech.

d) Test of Mouse Sera and Culture Supernatants for Detection of ProPSA in PCa Sera

For screening mouse sera, primary cultures and the cloned MABs for reaction with proPSA forms in PCa serum, microtiter plates coated with recombinant streptavidin (MicroCoat, Penzberg, Cat. No. 12-K 96 N, lot MC 289) were coated with 1 μg/ml biotinylated Fab fragment from the monoclonal antibody M-30 that only detects free PSA, in PBS plus 0.5% Crotein C (100 μl per well, 10 minutes incubation at room temperature while shaking), then washed 3× with 0.9% NaCl/0.05% TWEEN 20. Then incubation was carried out with 100 μl of the PCa serum undiluted or diluted 1:10 with PBS for 1 hour while shaking at room temperature. In the next step, 100 μl of the antibody solution to be investigated was added to a coated well and incubated for 1 hour at room temperature while shaking. After washing 3× with 0.9% sodium chloride/0.05% TWEEN 20, 100 μl of a POD-labeled Fab fragment of a polyclonal antibody from sheep to mouse-Fcγ (Roche Diagnostics GmbH, I.D. No. 1431323, corresponding to 25 mU/ml) was added in each case to detect the bound antibody in the sample, incubated for 1 hour at room temperature while shaking, then washed 3× with 0.9% sodium chloride/0.05% TWEEN 20.

Finally, 100 μl ABTS (Roche Diagnostics GmbH, Cat. No. 1204521 and 1204530) was added to each well. After 20 minutes at room temperature, absorbance was measured at 405/492 nm in an MR700 microplate reader from Dynatech. PSA-free serum from female donors was used as the control.

To check that the antibodies produced showed no reaction with free PSA from sperm, free PSA from Scripps, San Diego, Cat. No. P 0714, lot 98 43 649, 1 μg/ml, dissolved in PBS plus 0.5% Crotein C, was used as the antigen instead of the PCa sera. The results are summarized in Table 2.

TABLE 2
Reaction of a high-titer PCa serum containing PSA with sera
from mice immunized with proPSA peptide immunogen as
compared with a zero serum
Results [mE]

| Mouse # | Prostate Ca Serum | PSA-free Serum | PSA |
|---|---|---|---|
| 493-1 | 286 | 39 | 38 |
| 493-2 | 231 | 36 | 38 |
| 493-3 | 71 | 10 | 106 |
| 493-4 | 35 | 35 | 35 |
| 493-5 | 106 | 39 | 40 |
| 493-6 | 166 | 37 | 37 |
| 493-7 | 143 | 37 | 37 |
| 493-8 | 209 | 37 | 37 |
| 493-9 | 74 | 35 | 38 |
| Zero serum | 37 | 38 | 56 |

The results show, on the one hand, that most of the sera investigated from mice immunized as described above react specifically with the PCa serum containing PSA. Since, on the other hand, they do not react with free PSA from sperm that has a normal N-terminal of the mature PSA, these results also verify that proPSA forms must be present in this PCa serum.

Primary cultures were tested in analogous fashion, and cultures that showed a positive reaction with the proPSA peptide and the serum containing proPSA but that did not show a reaction with (sperm) PSA, cloning was carried out using a fluorescence-activated cell sorter in 96-well cell culture plates. Interleukin-6 (100 U/l) was added to the medium as a growth additive. The clones listed in Table 3 were obtained in this fashion.

TABLE 3
Clones with reactivity to proPSA

| Clone | IgG Subclass | Absorption/Test |
|---|---|---|
| 1.001.7 | IgG1 | 1.60 |
| 2.003.6 | IgG1 | 1.59 |
| 2.005.11 | IgG1 | 2.49 |
| 1.023.6 | IgG1 | 2.09 |
| 2.002.4 | IgG1 | 1.42 |

Obtaining Immunoglobulins from Mouse Ascites

The hybridoma cells obtained were used in a standard procedure to produce IgG by forming ascites in mice. These antibodies were purified from the ascites according to common protein-chemistry methods, e.g., according to Methods in Enzymology 121:587-695, 1986.

### EXAMPLE 3

Test for Specificity for [−5, −6, −7] ProPSA

As described in Example 2(d), proPSA from PCa serum was bound in wells of microtiter plates precoated with streptavidin. The proPSA antibodies, together with the proPSA peptides of different lengths, were then incubated together for 1 hour. The free peptides added compete in this step with proPSA from the PCa serum on the solid phase to bind to the antibodies. Peptides that are well-detected result in a displacement reaction and, therefore, a signal reduction, because fewer specific antibodies and, therefore, fewer detection antibodies, are bound. As shown in FIG. 2, only the [−6] proPSA and [−5] proPSA peptides lead to a strong displacement reaction. The [−7] proPSA was not run in this experiment. In other experiments, the [−7] proPSA peptide exhibited competition that was just as good as that of the [−5] or the [−6] proPSA peptide.

### EXAMPLE 4

Detection of [−5, −6, −7] ProPSA in an ELISA

Detection of [−5, −6, −7] proPSA was carried out in a sandwich ELISA with the proPSA MAB as the capture antibody and a POD-labeled MAB to free PSA as the detection reagent. MAB 1.023.6 was biotinylated using standard methods and used in a concentration of 2.5 μg/ml. This test was carried out like the Enzymun test on free PSA with the main difference being that the biotinylated MAB 1.023.6 according to the invention was used as the capture reagent.

### EXAMPLE 5

ROC Analyses with Different PSA Fractions

In 41 samples altogether having a total PSA value of between 4-10 ng/ml, but with a certain diagnosis with regard for BPH and PCa, the parameters of free PSA and total PSA were determined using the ELECSYS automated immunoanalyzer (Roche Diagnostics) and proPSA as described in Example 4. The quotients of free PSA to total PSA, proPSA to total PSA and free PSA to proPSA were calculated and subjected to an ROC analysis just like the individual parameters. As illustrated clearly in FIG. 3, the greatest AUC value results for the ratio of free PSA to proPSA. This parameter therefore makes a much better contribution to the differentiation of BPH and PCa than the two other relative values or the respective individual values.

What is claimed is:

1. An antibody specific for [−5, −6, −7] proPSA and essentially non-reactive with [−4] proPSA and shorter forms of proPSA.

2. An antibody specific for [−5, −6, −7] proPSA having less than 10% cross-reactivity with [−4] proPSA and shorter forms of proPSA.

3. The antibody according to claim 1 or 2, wherein the antibody is a monoclonal antibody.

4. An antibody having the same binding properties as an antibody secreted by the clone stored as ACC 2456 at the DSMZ.

5. A method for producing an antibody specific for [−5, −6, −7] proPSA comprising:
a) immunizing a host with an immunogen to produce an immunogenic response, the immunogen comprising a synthetic, carrier-bound peptide corresponding to the propeptide sequence of [−5], [−6] or [−7] proPSA,
b) screening the antibodies produced in step a) for an antibody that is essentially non-reactive with [−4] proPSA or shorter forms of proPSA, and
c) isolating and culturing the antibody from step b).

6. A method for producing an antibody specific for [−5, −6, −7] proPSA comprising:
a) immunizing a host with an immunogen to produce an immunogenic response, the immunogen comprising a synthetic, carrier-bound peptide corresponding to the propeptide sequence of [−5], [−6] or [−7] proPSA,
b) screening the antibodies produced in step a) for an antibody having less than 10% cross-reactivity with [−4] proPSA and shorter forms of proPSA, and
c) isolating and culturing the antibody from step b).

7. A method for determining [−5, −6, −7] proPSA in a sample comprising:
a) combining the sample with a capture antibody comprising an antibody specific for [−5, −6, −7] proPSA whereby the capture antibody binds with the [−5, −6, −7] proPSA in the sample and is essentially non-reactive with [−4] proPSA and shorter forms of proPSA, the capture antibody being bound directly or indirectly to a solid phase,
b) adding a detection antibody to the combination in step a) comprising an antibody specific for free PSA bound to a detectable label, whereby the detection antibody binds in sandwich fashion with the [−5, −6, −7] proPSA bound to the solid phase via the capture antibody, and
c) determining the presence or amount of labeled antibody bound in step b) as a measure of [−5, −6, −7] proPSA in the sample.

8. A method for determining [−5, −6, −7] proPSA in a sample comprising:
a) combining the sample with a capture antibody comprising an antibody specific for [−5, −6, −7] proPSA whereby the capture antibody binds with the [−5, −6, −7] proPSA in the sample and has less that 10% cross-reactivity with [−4] proPSA and shorter forms of proPSA, the capture antibody being bound directly or indirectly to a solid phase,
b) adding a detection antibody to the combination in step a) comprising an antibody specific for free PSA bound to a detectable label, whereby the detection antibody binds in sandwich fashion with the [−5, −6, −7] proPSA bound to the solid phase via the capture antibody, and
c) determining the presence or amount of labeled antibody bound in step b) as a measure of [−5, −6, −7] proPSA in the sample.

9. The method of claim 7 or 8, wherein the capture antibody is an antibody having the same binding properties as an antibody secreted by the clone stored as ACC 2456 at the DSMZ.

10. A diagnostic test composition for determining [−5, −6, −7] proPSA comprising an antibody specific for [−5, −6, −7] proPSA and essentially non-reactive with [−4] proPSA and shorter forms of proPSA.

11. A diagnostic test composition for determining [−5, −6, −7] proPSA comprising an antibody specific for [−5, −6, −7] proPSA and having less than 10% cross-reactivity with [−4] proPSA and shorter forms of proPSA.

12. The test composition of claim 10 or 11, wherein the antibody is a monoclonal antibody.

13. The test composition of claim 10 or 11, wherein the antibody has the same binding properties as an antibody secreted by the clone stored as ACC 2456 at the DSMZ.

14. An immunogen for producing an antibody specific for [−5, −6, −7] proPSA and essentially non-reactive with [−4] proPSA and shorter forms of proPSA, the immunogen comprising a peptide sequence selected from the group consisting of SEQ ID NOS. 1, 2 and 3, and an immunogenic carrier.

15. An immunogen for producing an antibody specific for [−5, −6, −7] proPSA and having less than 10% cross-reactivity with [−4] proPSA and shorter forms of proPSA, the immunogen comprising a peptide sequence selected from the group consisting of SEQ ID NOS. 1, 2 and 3, and an immunogenic carrier.

16. A method for diagnosing prostatic carcinoma in a patient comprising:
a) measuring the amount of [−5, −6, −7] proPSA in a blood, serum or plasma sample from the patient, and
b) correlating the amount of [−5, −6, −7] proPSA measured in step a) with the presence or absence of prostatic carcinoma in the patient.

17. A method for diagnosing prostatic carcinoma in a patient comprising:
a) measuring the amount of [−5, −6, −7] proPSA and total PSA in a blood, serum or plasma sample from the patient and
b) comparing the amount of [−5, −6, −7] proPSA to the amount of total PSA measured in step a), and
c) correlating the comparison obtained in step a) with the presence or absence of prostatic carcinoma in the patient.
